(19) United States
(12) Patent Application Publication
(10) Pub. No.: US 2013/0103003 A1
(43) Pub. Date: Apr. 25, 2013

(54) SECURE SYSTEM FOR TRANSFERRING MEDICAL FLUID

(71) Applicants: François CAPITAINE, ANGLET (FR); Frédéric Bonnet, (US)

(72) Inventors: François CAPITAINE, ANGLET (FR); Frédéric Bonnet, (US)

(73) Assignee: TECHNOFLEX, BIDART (FR)

(21) Appl. No.: 13/336,498

(22) Filed: Dec. 23, 2011

(30) Foreign Application Priority Data
Oct. 24, 2011 (FR) 11 59601

(51) Int. Cl. A61M 25/16

(52) U.S. Cl. 604/535

(57) ABSTRACT

A system for transferring medical fluid includes a male Luer connector and a female Luer connector, the male Luer connector including a collar surrounding at least part of a male conical portion whose outer surface is supported against the female conical connection surface of the female Luer connector during the assembly of the connectors. The maximum inside diameter at the entrance to the collar of the male Luer connector is strictly less than the smallest outside diameter of a standard female Luer connector or one according to ISO 594-2. The female Luer connector includes a collar surrounding at least part of the female conical connection, this collar having an inside diameter greater than or equal to the outside diameter of the collar of the male Luer connector, to engage and at least partially cover the collar of this male Luer connector during the assembly of the connectors.

This invention relates to the field of Luer connectors and particularly to a secure system for transferring medical solutions.

It is known to use Luer connectors to connect medical devices together for the use of transferring a medical solution, such as from a first container to a human body duct.

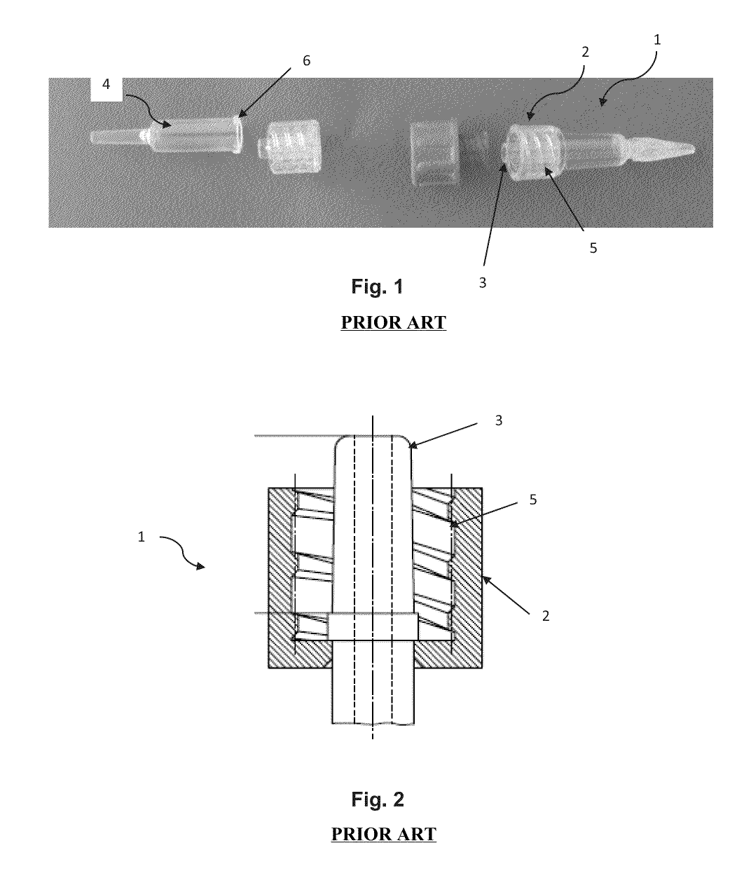

FIGS. 1 and 2 show male and female Luer connectors from the state of the art. A male Luer connector 1 also comprises a collar 2 surrounding at least part of a male conical portion 3 whose outer surface is supported against the female conical connection surface of the female Luer connector 4 during the assembly of these male 1 and female 4 Luer connectors.

The collar 2 of the male Luer connector 3 comprises an inner threading 5 while the female Luer connector 4 comprises at its end the largest of the lugs 6 to coordinate with the inner threading 5 of the collar 2 of the male Luer connector 1 with a view to their assembly.

The assembly of the two Luer connectors 1, 4 is obtained simply by application of a tightening torque by the operator. The application of this tightening torque, or twisting movement, thus leads, for example, to a movement that rotates the female Luer connector 4 around the inner threading 5 of the collar 2 of the male connector.

Luer connectors 1, 4 thus engaged are secured together, which prevents any accidental separation and external contamination of these Luer connectors, as has been defined by ISO 594-2 (International Organization for Standardization).

This standard also defines the exact dimensions for each of these Luer connectors.

Although these Luer connectors 1, 4 are easy to use, a patient recently died due to improper handling by the operator when coupling a large number of Luer connectors.

In particular, during the handling required for apheresis, the operator had reversed two anticoagulant and normal saline solution lines, which were fitted at the time with identical Luer connectors.

There is therefore an urgent need to change the connectors in the state of the art in order to avoid any new human handling error that could potentially put the patient's life at risk.

This invention aims to overcome this drawback by providing a system for transferring medical fluid that is especially simple in its design and in its operation and is aseptic and economical, allowing for a fully secure connection of the male and female Luer connectors.

To this end, the invention relates to a system for transferring medical fluid comprising a male Luer connector and a female Luer connector, the male Luer connector comprising a collar surrounding at least part of a male conical portion whose outer surface is supported against the female conical connection surface of the female Luer connector during the assembly of said male and female Luer connectors.

According to the invention,

the collar of the male Luer connector comprising an inside diameter and an outside diameter, the maximum inside diameter at the entrance to this collar is strictly less than the smallest outside diameter of a standard female Luer connector or one according to ISO 594-2,

the female Luer connector comprises a collar surrounding at least part of said female conical connection, the collar of the female Luer connector having an inside diameter that is greater than or equal to the outside diameter of the collar of the male Luer connector in order to become engaged and at least partially cover this collar of the male Luer connector during the assembly of said male and female Luer connectors.

In its most general embodiment, the disengagement of the male and female Luer connectors can be obtained either by applying a loosening torque or by applying an axial tensile force.

In the latter case, and purely as an example, the assembly of the male and female Luer connectors is obtained by nesting the female collar onto the male collar of the male Luer connector, the collar of the female Luer connector at least partially covering the collar of the male Luer connector.

In order to reinforce the assembly of these two connectors, the collars can be extended to present a larger contact surface and/or the surface of at least one of the two collars designed to come into contact with the surface of the other collar can be a rough surface in order to increase the friction between the two surfaces. The male collar can also have alternatively one or more annular projections to prevent the two Luer connectors from separating.

In accordance with ISO 594-2, the free inside diameter of the collar of the male Luer connector is therefore a maximum of 7.82 mm so as to reject any accidental insertion of a female Luer connector of the state of the art.

Preferably, the free inside diameter of the collar of the female Luer connector is then a maximum of 7.90+/−0.05 mm.

So as not to unduly limit the scope of its invention and to account for possible changes in the dimensions of the Luer connectors of the state of the art, the applicant does not wish to limit the inside diameter of its male Luer connector to a well-defined value.

In various particular embodiments of this system, each having its particular advantages and open to many possible technical combinations:
the system comprises means of attachment allowing said male and female Luer connectors to be secured during their assembly or after their assembly, at least a part of said means of attachment being designed to be placed outside of said male Luer connector.

These means of attachment are consequently placed outside of the defined inner volume between the collar of the male Luer connector and its male conical portion.

Purely as an example, these means of attachment may be brought back to the outside surface of the collar of the female Luer connector after the assembly of the male and female Luer connectors. This can then be an elastic band or a clamp collar, such as a Serflex (registered trademark) plastic collar.

For example, this clamp collar can thus comprise a belt with notches forming a first rack and a tack, said tack comprising on the one hand backstop gripping teeth and on the other hand a recess to ensure that this first rack comes into contact with said backstop gripping teeth, when said first rack is engaged in said tack, this contact blocking the position of said collar in the direction of the loosening of this collar.

Alternatively, these means of attachment are not brought back.

This can therefore broadly be an engagement structure such as that formed by a threading placed on a portion at least of the outer surface of the collar of the male Luer connector and a corresponding threading, or corresponding fins, placed on the inside surface of the collar of the female Luer connector. The inside threading or fins of the collar of the female Luer connector engage and coordinate with the outside threading of the collar of the male Luer connector during the assembly of the male and female Luer connectors.

These can also be lugs placed on the outside surface of the collar of the male Luer connector intended to be received and held by clipping into corresponding recesses forming protrusions from the collar of the female Luer connector.
the male conical portion comprises an inside channel whose diameter is at least equal to the diameter of the inside channel of the male conical portion of the male Luer connector defined by ISO 594-2.

Therefore, while being more compact and requiring less plastic during manufacturing, the male Luer connector according to this invention has a medical solution flow rate that is equal to the connectors of the prior art.

the collar of the female Luer connector may or may not be rotated around said female conical connection,

the collar of the male Luer connector, or respectively the female Luer connector, comprising at least one error-proofing element, the female Luer connector, or respectively the collar of said male Luer connector, comprises at least one corresponding unlocking body, said at least one unlocking body engaging and coordinating with said at least one error-proofing element to allow the assembly of said male and female Luer connectors during the application of a tightening torque, said error-proofing element prohibiting any assembly of the Luer connector provided with such an element with a corresponding Luer connector without said unlocking body,

the collar of the female Luer connector being rotatable, the unlocking body placed on the outside surface of the female conical connection also forms a guide element in translation such that, during the application of a tightening torque, said collar rotates while said female conical connection moves in translation,

said at least one unlocking body being a guiding groove in translation placed on the outside surface of said female conical connection, said at least one error-proofing element is a protrusion placed on the inside surface of the collar of said male Luer connector, said protrusion being intended to engage at least partly into said groove.

The female Luer connector comprises at least two guiding grooves in translation placed on the outside surface of said female conical connection, said grooves being diametrically opposed relative to said female conical connection.

the collar of said male Luer connector comprising an outside threading and said female Luer connector comprising two error-proofing elements, each of said error-proofing elements is a flexible tab having at its end a fin, or lug, designed to engage and coordinate with the outside threading of the collar of said male Luer connector during the application of a tightening torque, said error-proofing elements being placed so as to prevent access to said female conical connection at rest while being able to be plated at least partially against the inside wall of said collar to open up access to said female conical connection during the assembly of said male and female Luer connectors,

at least one of said male and female Luer connectors comprises a conical end connected such that it may or may not be separable from said collar and in fluid communication with it.

Advantageously, each connector can also comprise a separable sealing element to ensure that it is aseptic. This sealing element also seals the free end from the conical end.

The invention also relates to a male Luer connector intended to be used in a fluid transfer system as described above, said male Luer connector comprising a threading designed to engage and coordinate with the corresponding threading or the corresponding fins of a female Luer connector with a view to their assembly and a male conical portion whose outside surface is supported against the female conical connection surface of said female Luer connector during the assembly of said male and female Luer connectors.

According to the invention,

said male Luer connector comprises a collar with an outside surface and an inside surface, said threading being placed on at least one portion of the outside surface of said collar,

the maximum inside diameter at the entrance to said collar is strictly less than the smallest outside diameter of a female Luer connector according to ISO 594-2.

In accordance with ISO 594-2, the free inside diameter of the collar of the male Luer connector is therefore a maximum of 7.82 mm so as to reject any accidental insertion of a female Luer connector of the state of the art.

Advantageously, said male conical portion comprises an inside channel whose diameter is defined by ISO 594-2 so as to keep the fluid flow rate constant relative to the male Luer connectors of the state of the art.

Preferably, this male Luer connector is made from a single piece of plastic.

The invention also provides a female Luer connector intended to be used in the fluid transfer system as described above, this female Luer connector comprising:

a collar comprising inside fins, or an inside threading, intended to engage and coordinate with a threading placed on at least one portion of the outside surface of the collar of a male Luer connector during the application of a tightening torque,

said female Luer connector comprising a female conical connection whose inside surface is supported against the outside surface of the male conical portion of said male Luer connector during the assembly of said male Luer connector and said female Luer connector.

Preferably, the collar of this female Luer connector comprising inside fins, the diameter of this collar taken at the base of said fins is at least equal, to within +0.1 mm, to the diameter of the collar of said male Luer connector taken between its outside threading.

The invention also relates to a medical device comprising at least one container containing a medical fluid used with a medical fluid transfer system as described above.

The invention also relates to a medical device comprising a syringe equipped with a male Luer connector or a female Luer connector as described above.

Finally, the invention relates to a system for transferring medical fluid comprising a male Luer connector and a female Luer connector, said male Luer connector comprising a collar surrounding at least part of a male conical portion whose outer surface is supported against the female conical connection surface of said female Luer connector during the assembly of said male and female Luer connectors.

According to the invention,

said female Luer connector comprises a collar surrounding at least part of said female conical connection, said collar having an inside diameter that is greater than or equal to the outside diameter of the collar of said male Luer connector in order to become engaged and at least partially cover said collar of said male Luer connector during the assembly of said male and female Luer connectors,

the collar of said male Luer connector, or respectively the female Luer connector, comprising at least one error-proofing element, said female Luer connector, or respectively the collar of said male Luer connector, comprises at least one corresponding unlocking body, said at least one unlocking body engaging and coordinating with said at least one error-proofing element to allow the assembly of said male and female Luer connectors during the application of a tightening torque, said error-proofing element prohibiting any assembly of the Luer connector provided with such an element with a corresponding Luer connector without said unlocking body.

The inside diameter of the collar of the male Luer connector can thus be equal to the diameter of the collar of a standard male Luer connector or one according to ISO 594-2.

Although the error-proofing element(s), or the unlocking body(ies), of the male Luer connector must be located on the collar, these corresponding elements or bodies can be located on the collar and/or on the outside surface of said female conical connection for the female Luer connector.

The invention will be described in more detail with reference to the accompanying drawings, in which:

FIG. 1 is a view of a male Luer connector/female Luer connector assembly according to one embodiment of the prior art;

FIG. 2 is a partial cross-sectional view of the male Luer connector from FIG. 1,

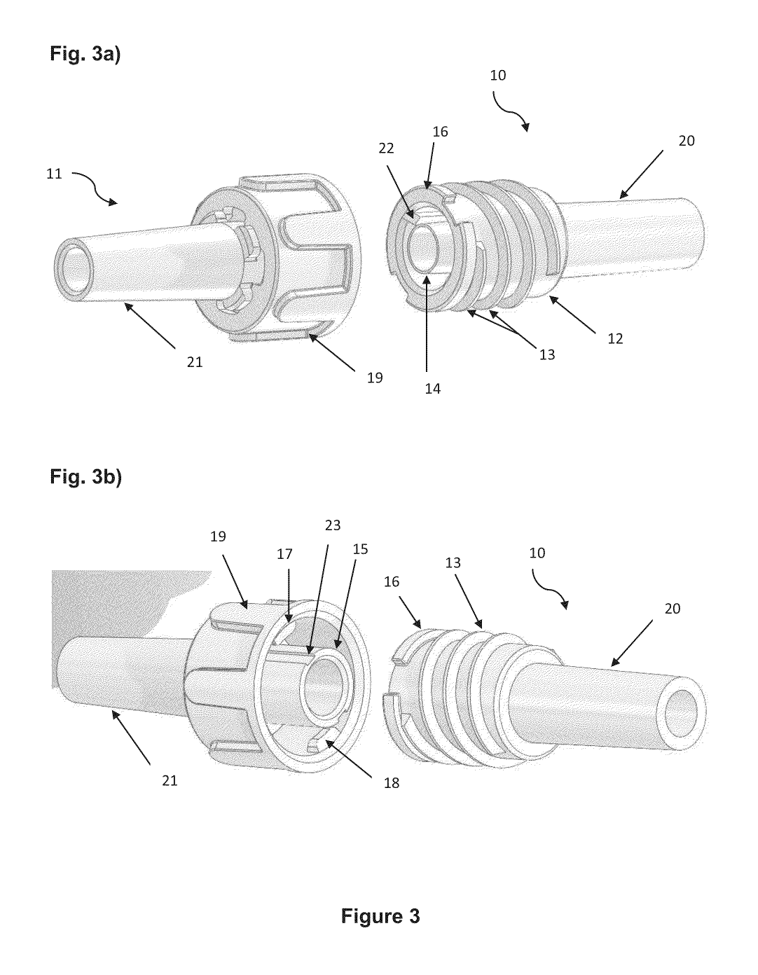

FIG. 3 is a profile view of a system for transferring medical fluid comprising a male Luer connector and a female Luer connector, according to a first embodiment of the invention, FIG. 3a) showing the male conical portion of the male Luer connector and FIG. 3b) showing the female conical connection of the female Luer connector,

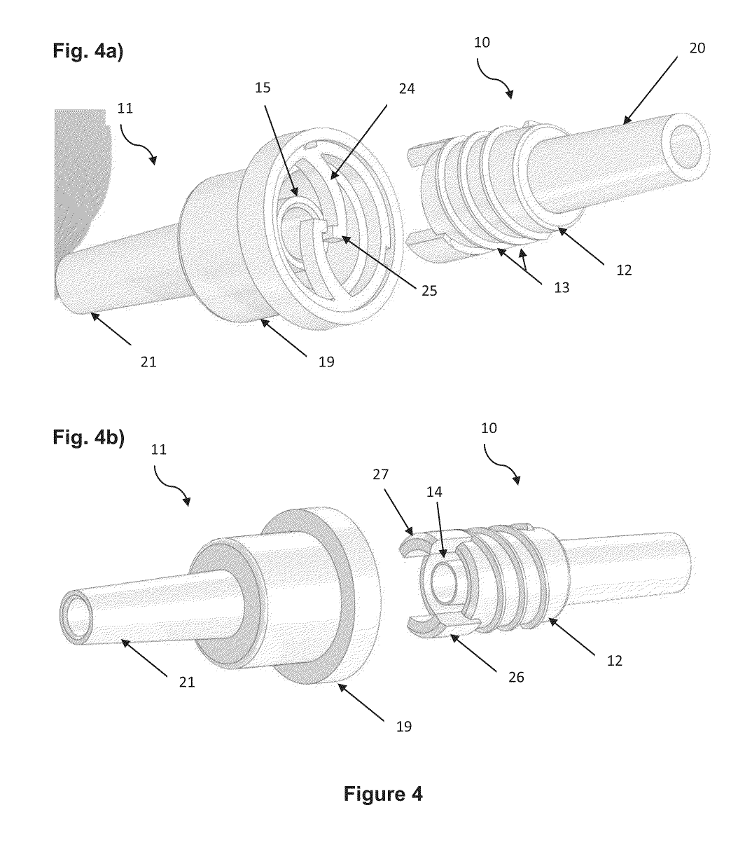

FIG. 4 is a profile view of a system for transferring medical fluid comprising a male Luer connector and a female Luer connector, according to a second embodiment of the invention, FIG. 4a) showing the female conical connection of the female Luer connector and FIG. 4b) showing the male conical portion of the male Luer connector.

FIG. 3 is a schematic representation of a system for transferring medical fluid according to a first embodiment of the invention.

This transfer system comprises a male Luer connector 10 and a female Luer connector 11 intended to be assembled to allow fluid to be transferred by applying a tightening torque.

The male Luer connector 10 comprises a fixed collar, or a skirt, 12 having an outside surface with a threading 13. This collar 12 surrounds a male conical portion 14 whose outside surface is supported against the female conical connection surface 15 of the female Luer connector 11 during the assembly of these two Luer connectors.

The collar 12 of the male Luer connector having an inside diameter and an outside diameter, the maximum inside diameter at the entrance of this collar, meaning at the end of this collar allowing the female conical connector 15 to be inserted, is strictly less than the smallest outside diameter of a standard female Luer connector or one according to ISO 594-2. Therefore, an operator having to connect multiple connector pairs advantageously cannot mistakenly connect a female Luer connector of the state of the art with the male Luer connector of the invention.

In accordance with the current ISO 594-2, the free inside diameter of the collar of the male Luer connector is therefore a maximum of 7.82 mm.

Advantageously, the male conical portion 14, whose axis of symmetry coincides with the axis of symmetry of the collar 12 of the male connector, defines an inside channel for the passage of medical fluid to be transferred. The diameter of this inside channel is at least equal to the diameter of the inside channel of the male conical portion of the male Luer connectors of the state of the art in order to keep a flow rate of solution that is at least similar or even greater.

The end of the collar 12 of the male Luer connector 10 also has a non-continuous crown, or pad, 16 that defines the entry points for possibly accessing the outside threading 13 of the collar of the male Luer connector 10. These entry points can only be crossed by fins, or lugs, 17, 18, with a complementary shape placed on the inside of the movable collar 19 of the female Luer connector, these fins 17, 18 being designed to engage in the outside threading 13 of the collar 12 of the male Luer connector 10 to allow the movable collar 19 of the female Luer connector 11 to rotate around this threading 13 during the application of a tightening torque.

The male Luer connector 10 is a single piece and obtained here by injection molding of a medically inert thermoplastic material. It can, purely as an example, be made of a polyolefin (such as polyethylene, polypropylene, a homopolymer, or a polypropylene copolymer) or a polyolefin comprising a thermoplastic elastomer (TPE) such as SEB (styrene-ethylene-butylene block copolymer) or SEBS (styrene-ethylene-butylene-styrene block copolymer), etc.

The fixed collar 12 of the male Luer connector 10 is also connected to a conical end 20 that is designed to be inserted into a duct, such as a tubing (not shown). This conical end 20 is connected such that it may or may not be separable from the collar 12 and in fluid communication with the inside channel of the male conical portion 14.

The collar 19 of the female Luer connector 11 comprises two inside fins, or inside lugs, 18, 19 arranged opposite one another on the inside wall of the collar 19 so as to engage in the entry points defined by the non-continuous pad 16 of the collar 12 of the male Luer connector during the coupling of the male 10 and female Luer connectors 11. These lugs 18, 19, having passed this non-continuous pad 16, can then engage and coordinate with the threading 13 placed on the outside surface of the collar 12 of the male Luer connector 10 during the application of a tightening torque.

The collar 19 of the female Luer connector 11 can be rotated around the female conical connection 15, which is connected to a conical end 21 designed to be inserted into a tubing. The female conical connection 15 is positioned coaxially with the collar 19 of the female Luer connector 11.

In addition, the collar 12 of the male Luer connector comprises two internal error-proofing elements 22, meaning two error-proofing elements positioned on the inside surface of the collar 12 in the space separating this collar from the male conical portion 14 of the male Luer connector 10. These two error-proofing elements 22 allow the coupling of the male Luer connector 10 only with a female Luer connector 11 comprising two corresponding unlocking bodies 23. Each unlocking body 23 is thus designed to engage and coordinate with one of the error-proofing elements 22 to allow the assembly of the male and female Luer connectors during the application of a tightening torque.

The error-proofing elements 22 here are protrusions whose right section is rectangular, and the unlocking bodies 23 are grooves whose shape corresponds to the protrusions, these grooves being positioned on the outside surface of the female conical connection 15.

FIG. 4 is a profile view of a system for transferring medical fluid comprising a male Luer connector and a female Luer connector, according to a second embodiment of the invention. The elements of FIG. 4 having the same reference numbers as the elements of FIG. 3 represent the same objects, which will not be described again below.

The system for transferring fluid in FIG. 4 differs from what is described above in that the female Luer connector 11 comprises two error-proofing elements, each of these error-proofing elements consisting of a flexible tab 24 having at its end a fin, or lug, 25 designed to engage and coordinate with the outside threading 13 of the collar 12 of the male Luer connector during the application of a tightening torque.

These error-proofing elements 24, 25 are positioned so as to prevent access to the female conical connection 15 in a rest position while being able to be plated at least partially against the inside wall of the fixed collar 19 of the female Luer connector 11 to open up access to this female conical connection 15 positioned for the assembly of the male Luer connector and the female Luer connector. Some of these error-proofing elements 24, 25 are therefore positioned in front of the female conical connection in order to form a screen preventing access to this female conical connection 15.

The male Luer connector 10 comprises unlocking bodies 26, 27 to unlock the error-proofing elements 24, of the female Luer connector 11 to form a seal between the male conical portion 14 and the female conical connection 15.

These unlocking bodies 26, 27 here are projections protruding from the collar 12 of the male Luer connector 10. These curved projections 26, 27 are spaced apart from one another so that they can be inserted into said error-proofing elements 24, 25 and the fixed collar 19 of the female Luer connector 11 to engage in this collar 19.

These unlocking bodies 26, 27 then come to be supported against the flexible tabs 24 during the application of a tightening torque so as to lead the free part of these lugs 24 and to plate it against the inside wall of the collar 19 of the female Luer connector, thereby opening up access to the female conical connection 15.

Because the collar 19 of the female Luer connector 11 has a hollow cylindrical shape, these flexible tabs 24 are rounded to be able to be fully plated against the inside wall of the collar 19 and to thereby fully open up access to the female conical connection 15.

1. A system for transferring medical fluid comprising a male Luer connector (10) and a female Luer connector (11), said male Luer connector (10) comprising a collar (12) surrounding at least part of a male conical portion (14) whose outer surface is supported against the female conical connection surface (15) of said female Luer connector (11) during the assembly of said male and female Luer connectors, characterized in that
the collar (12) of said male Luer connector (10) comprising an inside diameter and an outside diameter, the maximum inside diameter at the entrance to said collar (12) is strictly less than the smallest outside diameter of a standardiscd female Luer connector (11) according to ISO 594-2,
said female Luer connector (11) comprises a collar (19) surrounding at least part of said female conical connection (15), said collar having an inside diameter that is greater than or equal to the outside diameter of the collar (12) of said male Luer connector (10) in order to become engaged and at least partially cover said collar (12) of said male Luer connector (10) during the assembly of said male and female Luer connectors.

2. A system according to claim 1, characterized in that it comprises means of attachment (12, 13, 16, 17) allowing said male (10) and female Luer connectors (11) to be secured during their assembly or after their assembly, at least a part of said means of attachment being designed to be placed outside of said male Luer connector (10).

3. A system according to claim 1, characterized in that said male conical portion (14) comprises an inside channel whose diameter is at least equal to the diameter of the inside channel of the male conical portion (14) of the male Luer connector (10) defined by ISO 594-2.

4. A system according to claim 1, characterized in that the collar of said female Luer connector (11) can be rotated around said female conical connection (15).

5. A system according to claim 1, characterized in that the collar (12) of said male Luer connector (10), or respectively the female Luer connector (11), comprising at least one error-proofing element (22, 24, 25), said female Luer connector (11), or respectively the collar (12) of said male Luer connector (10), comprises at least one corresponding unlocking body (23, 26, 27) said at least one unlocking body (23, 26, 27) engaging and coordinating with said at least one error-proofing element (22, 24, 25) to allow the assembly of said male (10) and female Luer connectors (11) during the application of a tightening torque, said at least one error-proofing element (22, 24, 25) prohibiting any assembly of the Luer connector provided with at least one such element with a corresponding Luer connector without said at least one unlocking body (23, 26, 27).

6. A system according to claim 5, characterized in that, the collar (19) of said female Luer connector (11) being rotatable, said unlocking body placed on the outside surface of the female conical connection (15) also forms a guide element (23) in translation such that, during the application of a tightening torque, said collar (19) rotates while said female conical connection (15) moves in translation.

7. A system according to claim 6, characterized in that said at least one unlocking body being a guiding groove (23) in translation placed on the outside surface of said female conical connection (15), said at least one error-proofing element (22) is a protrusion placed on the inside surface of the collar of said male Luer connector (10), said protrusion being intended to engage at least partly into said groove.

8. A system according to claim 7, characterized in that it comprises at least two guiding grooves (23) in translation placed on the outside surface of said female conical connection (15), said grooves (23) being diametrically opposed relative to said female conical connection (15).

9. A system according to claim 5, characterized in that the collar (12) of said male Luer connector (10) comprising an outside threading (13) and said female Luer connector (11) comprising two error-proofing elements, each of said error-proofing elements is a flexible tab (24) having at its end a lug (25) designed to engage and coordinate with the outside threading (13) of the collar of said male Luer connector (10) during the application of a tightening torque, said error-proofing elements (24, 25) being placed so as to prevent access to said female conical connection (15) at rest while being able to be plated at least partially against the inside wall of said collar (19) to open up access to said female conical connection (15) during the assembly of said male and female Luer connectors.

10. A system according to claim 9, characterized in that said unlocking bodies are projections (26, 27) protruding from the collar (12) of said male Luer connector (10) being spaced apart from one another so as to be inserted between said error-proofing elements (24, 25) to engage into the collar (19) of said female Luer connector (11), said unlocking bodies then coming to be supported against said flexible tabs (24) during the application of a tightening torque so as to plate at least partially said tabs (24) against the inside surface of said collar (19), thereby freeing up access to said female conical connection (15) of said female Luer connector (11).

11. A system according to claim 9, characterized in that, the collar of said female Luer connector (11) having a hollow cylindrical shape, said flexible tabs (24) are rounded so as to be able to be fully plated against the inside wall of said collar.

12. A system according to claim 1, characterized in that at least one of said male and female Luer connectors comprises a conical end (20, 21) connected such that it may or may not be separable from said collar and in fluid communication with it.

13. A male Luer connector (10) intended to be used in the fluid transfer system according to claim 1, said male Luer connector (10) comprising a threading (12) designed to engage and coordinate with the corresponding threading or the corresponding fins of a female Luer connector (11) with a view to their assembly and a male conical portion (14) whose outside surface is supported against the female conical connection surface (15) of said female Luer connector (11) during the assembly of said male and female Luer connectors, characterized in that
said male Luer connector (10) comprises a collar (12) with an outside surface and an inside surface, said threading (13) being placed on at least one portion of the outside surface of said collar (12),
the maximum inside diameter at the entrance to said collar (12) is strictly less than the smallest outside diameter of a female Luer connector (11) according to ISO 594-2.

14. A connector according to claim 13, characterized in that it is made from a single piece of plastic.

15. A female Luer connector (11) intended to be used in the fluid transfer system according to claim 1, said female Luer connector (11) comprising:
a collar (19) comprising inside fins (18), or an inside threading, intended to engage and coordinate with a threading (13) placed on at least one portion of the outside surface of the collar (12) of a male Luer connector (10) during the application of a tightening torque,
said female Luer connector (11) comprising a female conical connection (15) whose inside surface is supported against the outside surface of the male conical portion (14) of said male Luer connector (10) during the assembly of said male Luer connector (10) and said female Luer connector (11).

16. A connector according to claim 15, characterized in that, said collar (19) comprising inside fins, the diameter of said collar taken at the base of said fins is at least equal, to within +0.1 mm, to the diameter of the collar (12) of said male Luer connector (10) considered between its outside threading (13).

17. A medical device comprising at least one container containing a medical fluid used with a medical fluid transfer system according to claim 1.

18. A system according to claim 2, characterized in that said male conical portion (14) comprises an inside channel whose diameter is at least equal to the diameter of the inside channel of the male conical portion (14) of the male Luer connector (10) defined by ISO 594-2.

19. A system according to claim 2, characterized in that the collar of said female Luer connector (11) can be rotated around said female conical connection (15).

20. A system according to claim 3, characterized in that the collar of said female Luer connector (11) can be rotated around said female conical connection (15).
